(19) United States
(12) Patent Application Publication
(10) Pub. No.: US 2023/0346793 A1
(43) Pub. Date: Nov. 2, 2023

(54) Compositions and Methods for Treating SARS-CoV-2

(71) Applicants: Mickey Hu, Palo Alto, CA (US); Bo Yu, Sunnyvale, CA (US)

(72) Inventors: Mickey Hu, Palo Alto, CA (US); Bo Yu, Sunnyvale, CA (US)

(21) Appl. No.: 17/999,448

(22) PCT Filed: May 25, 2021
(86) PCT No.: PCT/US21/34050

§ 371 Date: Nov. 21, 2022

Related U.S. Application Data
(60) Provisional application No. 63/031,463, filed on May 28, 2020.

(51) Int. Cl. A61K 31/5415; A61K 31/40; A61K 31/706; A61K 45/06; A61P 31/14

(52) U.S. Cl. CPC A61K 31/5415; A61K 31/40; A61K 31/706; A61K 45/06; A61P 31/14

(57) ABSTRACT

The present disclosure provides compositions and methods for treating SARS-CoV-2. Bepridil and Trifluoperazine effectively inhibit SARS-CoV-2 infections. The EC₅₀ values for Bepridil and Trifluoperazine were 7.13 and 7.7 μM, respectively. Moreover, using drug combinations with Remdesivir significantly potentiate Bepridil-mediated inhibition of SARSCoV-2 infections.

### BACKGROUND

The presently disclosed subject matter provides compositions and methods for treating the severe acute respiratory syndrome coronavirus 2 (SARS-CoV-2), which is currently the biggest public health challenge to the biomedical community of the last century. Despite multiple public health measures (Colbourn T, 2020; Cowling B J, et al., 2020; Prem K, et al., 2020), there remains an urgent unmet need for effective pharmacological therapies to treat SARS-CoV-2-infected patients and minimize mortality, decrease pressures on intensive care units and health systems, and optimally decrease subsequent viral transmission.

In the past two decades, SARS coronavirus (SARS-CoV) and Middle East respiratory syndrome coronavirus (MERS-CoV) were transmitted from animals to humans, causing severe respiratory diseases SARS and MERS in endemic areas. In December 2019, another coronavirus was discovered in patients with infectious respiratory disease in Wuhan, Hubei province, China, to have the ability for human-to-human transmission. The disease, now termed coronavirus disease 2019 (COVID-19), has spread rapidly all over the world, resulting in a pandemic. COVID-19 is induced by the pathogenic SARS-CoV-2 and is associated with more than 5.1 million confirmed cases and 330,000 deaths have been recorded worldwide, and more than 1.6 million cases and over 95,000 deaths in the US as of May 23, 2020 (COVID-19 Map Johns Hopkins University and Medicine). The major phenotype of COVID-19 is severe acute respiratory distress syndrome (ARDS), just like the diseases caused by SARS-CoV and MERS-CoV (de Wit E, et al., 2016; Huang C, et al., 2020).

SARS-CoV-2 enter the host cells through a cellular receptor termed angiotensin-converting enzyme-2 (ACE2), which is a type I transmembrane metallopeptidase with an extracellular ectodomain containing its zinc-coordinating catalytic site (Tipnis S R, et al., 2000; Donoghue M, et al., 2000). It has been well documented that ACE2 is a key receptor for the SARS-coronaviruses (Li W, et al., 2003).

### SUMMARY

The disclosure describes compositions and methods for the treatment of the SARS-CoV, MERS, SARS-CoV-2 virus-induced COVID-19, or other related viral diseases. The compositions include several FDA-approved small-molecule drugs that significantly inhibit the Akt signaling pathway. These compositions can inhibit SARS-CoV, MERS, or SARS-CoV-2 viral replication in SARS-CoV, MERS, or SARS-CoV-2 infected cells. These compositions can also potently activate p53 tumor suppressor, which had can also lead to inhibition of SARS-CoV, MERS, or SARS-CoV-2 replication. The compositions can include Bepridil and/or Trifluoperazine. Bepridil and/or Trifluoperazine can treat SARS-CoV, MERS, or SARS-CoV-2 infections because both inhibit replication of SARS-CoV, MERS, or SARS-CoV-2.

The disclosure also describes treatment methods using the described compositions to treat subjects infected with SARS-CoV, MERS, or SARS-CoV-2, or prophylactically treat subjects at risk for SARS-CoV, MERS, or SARS-CoV-2 infection. The methods also include treatment of subjects who have been exposed to SARS-CoV, MERS, or SARS-CoV-2, or who may have been exposed to SARS-CoV, MERS, or SARS-CoV-2. The anti-SARS-CoV-2 compositions can be formulated for administration to subjects including, for example, as an oral formulation, an injectable formulation, a nasal formulation, a sublingual formulation, a buccal formulation, a suppository formulation, a transdermal formulation, an inhalation formulation, a mucosal formulation, etc. Oral formulations can include immediate release formulations, controlled releases formulations, sustained release formulations, extended release formulations, etc. Injectable formulations can be prepared for delivery by any parenteral route including, for example, intravenous, subcutaneous, intramuscular, intradermal, intradural, pulmonary, etc.

The methods of treatment described herein can involve a single administration of a dosage form, or multiple administrations of a dosage form over a suitable time period. For example, the compositions described herein can be formulated for administration once a day, or multiple times a day (e.g., 2, 3, or 4 times per day), or for administration once a week, or once every 2, 3, 4, 5, of 6 weeks, or once every month, or every 2, 3, 4, 5, or six months.

The compositions described herein can be administered to a subject by any suitable means including, for example, orally, intravenously, subcutaneously, intramuscularly, intradermally, intradurally, nasally, sublingually, buccally, anally, intraperitoneally, etc.

The compositions described herein also include cocktails, or combinations of drugs that have anti-SARS-CoV-2 activity. The drugs in the cocktail can act by the same mechanism of action (targeting the same or similar target), or can act through different mechanisms of action leading to potential synergistic effects that can increase the effectiveness of the treatment. The drug cocktail or combinations described herein can also have the benefit of reducing escape of SARS-CoV-2 from therapy as multiple mutations may be necessary to escape the drug cocktail or combination when the different drugs act through different mechanisms of action.

The combination therapies described herein can also combine an antiviral agent with another therapeutic compound. The other therapeutic compound can be another antiviral, or a non-antiviral drug that ameliorates a symptom of a SARS-CoV, MERS, or SARS-CoV-2 infection. Such other therapeutic compound includes, for example, an analgesic, a decongestant, an anti-inflammatory, an immunosuppressive agent, etc.

### BRIEF DESCRIPTION OF THE FIGURES

FIG. 1. illustrates the compositions and structures for Bepridil and Trifluoperazine.

FIG. 2. shows Bepridil (BPD) exerting potent inhibitory activity against SARS-CoV-2 infections by plaque reduction assays. (A) The scheme of the experimental procedure is shown. (B) VeroE6 monkey cells (ATCC) were seeded in 24-well plates (5×10⁴ cells/well) in Dulbecco's Modified Eagle Medium (DMEM) supplemented with 1% non-essential amino-acid, 10 mM HEPES and 10% fetal bovine serum plus 1% Pen/Strep at 37° C., 5% CO2. 24 hours post-seeding, control, BPD, and other compound (X01) were added into culture medium at 37° C. After 1 hour, VeroE6 were infected with human SARS-CoV-2 (40-200 plaque forming units (PFU)) for 1 hour followed by washing with PBS once, the monolayer cells were overlaid with 1 ml medium containing 1% (w/v) of methylcellulose plus control (DMSO) or a compound (BPD or X01). After 5-day incubation, cells were washed with PBS and fixed with 250 μl of 4% formaldehyde for 30 min at room temperature. Cells were then stained with 300 μl of 0.5% of crystal violet for 5 min, and washed twice. After the plates were air dried, plaques were counted. The inhibition was calculated as the percentage of the mean of plaques reduced for the three drug wells as compared to the mean of the plaque counts for the three virus only wells in the same assay. (C) A BPD dose-dependent analysis on inhibiting SARS-CoV-2 infection was performed as described above. The data of this experiment indicated that the effective concentration of BPD that gave half-maximal response (EC₅₀) was 7.13 μM. **, P<0.005, *, P<0.05.

FIG. 3. shows Trifluoperazine (TFP) having potent inhibitory activity against SARS-CoV-2 infection. Plaque reduction assays for TFP were performed as described in the legend to FIG. 3. The data of a TFP dose-dependent analysis indicated that the EC₅₀ of TFP was 7.7 μM. ***, P<0.001, **, P<0.005, *, P<0.05.

FIG. 4. shows Remdesivir can potentiate the BPD-mediated inhibitory activity against SARSCoV-2 infection. Remdesivir (RDV) is an FDA-approved compound against SARS-CoV-2 infection recently. The combinations of BPD and various concentrations of RDV as indicated were examined by plaque reduction assays as described in the legend to FIG. 3. **, P<0.005, *, P<0.01.

FIG. 5. shows a diagram representing the model for the inhibition of SARS-CoV-2 infection by BPD and TFP. It has been shown that BPD and TFP inhibit calmodulin (Winkler M A, et al., 1987; Zimmer and Hofmann, 1984; Reiermann and Rüegg, 1986). Calmodulin interacts with the cytoplasmic domain of ACE2 and prevents shedding of the ACE2 ectodomain (Lambert D W, et al., 2007), which is a key receptor for the SARS-coronaviruses (Li W, et al., 2003). A schematic shows that BPD and TFP inhibit calmodulin and may lead to suppression of SARS-CoV-2 binding to ACE2 on the surface of target cells. In addition, BPD and TFP suppress phosphor-Akt (pAkt) and induce FOXO3 and p53 (FOXO3/p53) translocation from the cytoplasm into the nucleus, where activated p53-pS15 inhibits SARS-CoV-2 replication and may result in inhibition of SARS-CoV-2 infections.

### DETAILED DESCRIPTION

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

As used in this specification and the appended claims, the singular forms “a”, “an” and “the” include plural referents unless the context clearly indicates otherwise. Thus, for example, reference to “a polypeptide” includes more than one polypeptide.

The section headings used herein are for organizational purposes only and not to be construed as limiting the subject matter described.

### Definitions

The term “therapeutically effective amount” means an amount of a compound or combination of compounds that treats a disease; ameliorates, attenuates, or eliminates one or more symptoms of a particular disease; or prevents or delays the onset of one of more symptoms of a disease.

The terms “treating”, “treat” or “treatment” include preventative (e.g., prophylactic) and palliative treatment.

The term “pharmaceutically acceptable salts” includes the salts of compounds that are, within the scope of sound medical judgment, suitable for use with patients without undue toxicity, irritation, allergic response, and the like, commensurate with a reasonable benefit/risk ratio, and effective for their intended use, as well as the zwitterionic forms, where possible, of the compounds.

The term “salts” refers to inorganic and organic salts of compounds. These salts can be prepared in situ during the final isolation and purification of a compound, or by separately reacting a purified compound with a suitable organic or inorganic acid or base, as appropriate, and isolating the salt thus formed. Representative salts include the hydrobromide, hydrochloride, sulfate, bisulfate, nitrate, acetate, oxalate, palmitiate, stearate, laurate, borate, benzoate, lactate, phosphate, tosylate, besylate, esylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, for example, S. M. Berge, et al., “Pharmaceutical Salts,” J Pharm Sci, 66:1-19 (1977).

### Methods for Treating SARS-CoV-2

The compositions described herein significantly inhibit the Akt signaling pathway which is activated in response to SARS-CoV-2 viral replication in SARS-CoV-2 infected cells. These small-molecule drugs can also activate p53 tumor suppressor which can significantly inhibit replication of SARS-CoV-2. The compositions described herein can include Bepridil and/or Trifluoperazine which both potently inhibit Akt and calmodulin activities.

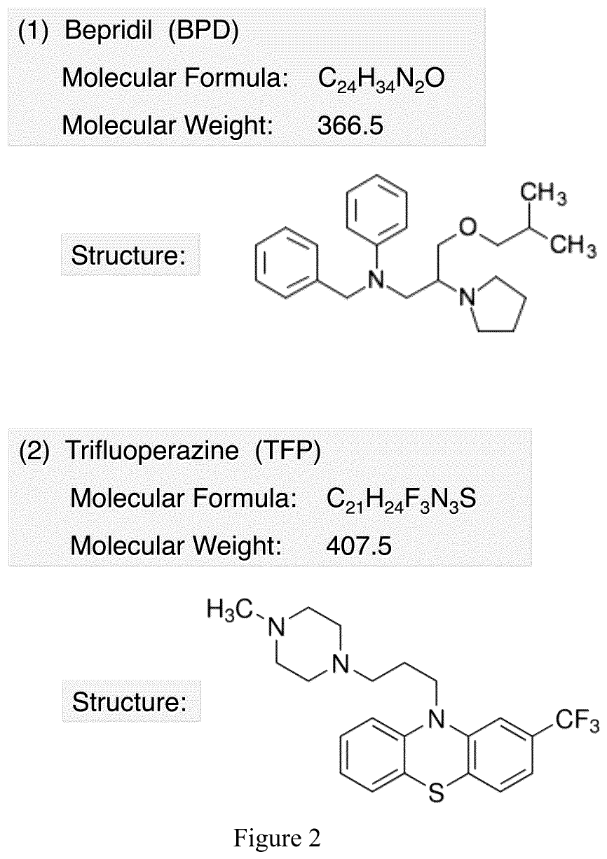

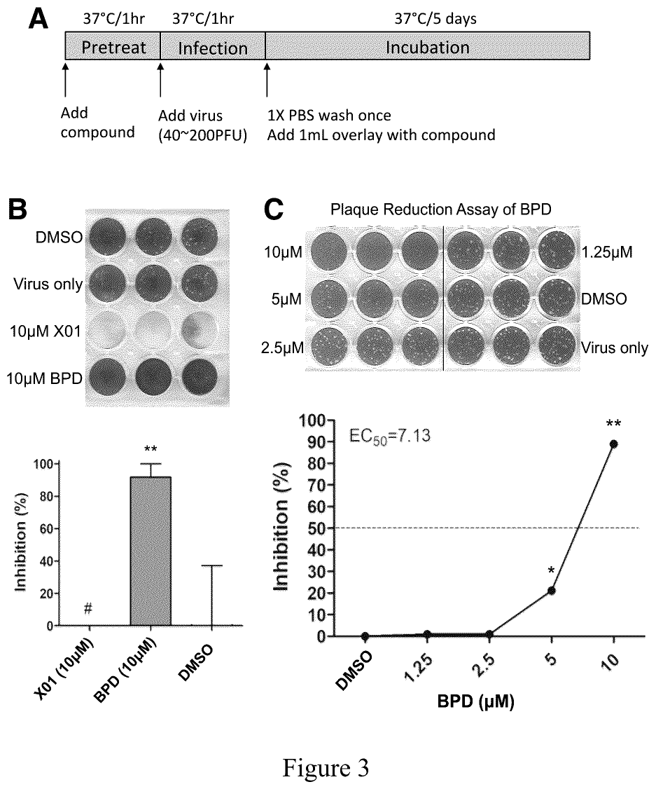

_Bepridil (BPD), analogs of BPD, derivatives of BOD, Trifluoperazine (TFP), analogs of TFP, and/or derivatives of TFP can effectively suppress SARS-CoV-2 replication, and so can be used to treat SARS-CoV-2 infections. Bepridil exerted potent inhibitory activity against SARS-CoV-2 infection in plaque reduction assays. Bepridil strongly inhibits infection by SARS-CoV-2 in a VeroE6 cell-based plaque reduction assay in a dose-dependent manner (FIG. 2). The respective EC₅₀ value for BPD was 7.13 μM. Similarly, Trifluoperazine also potently inhibited SARS-CoV-2 infection in a VeroE6 cell-based plaque reduction assay in a dose-dependent manner (FIG. 3). The respective EC₅₀ value for TFP was 7.7 μM.

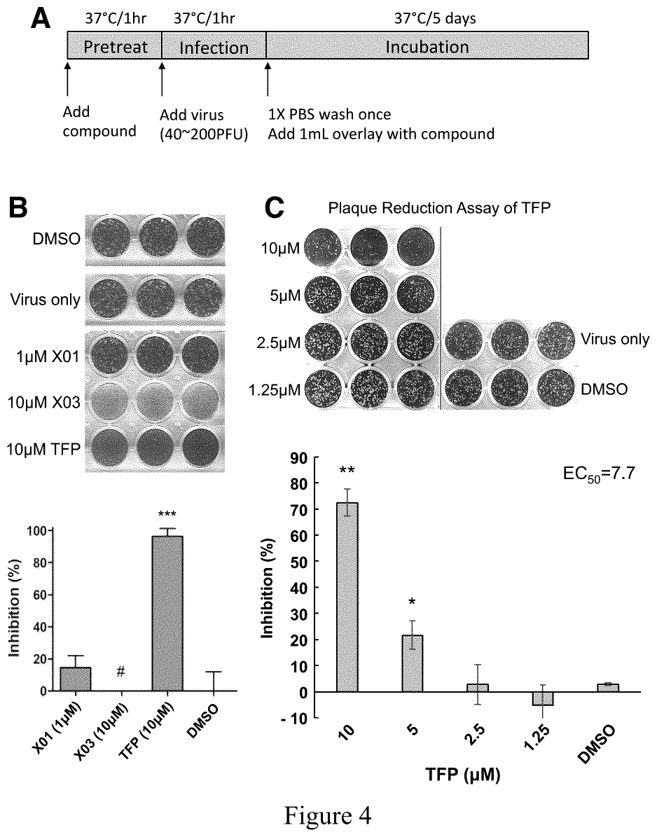

Remdesivir (RDV) is being used for the treatment of severe COVID-19 patients. RDV is an adenosine nucleotide analogue that is a broad-spectrum antiviral medication developed by Gilead Sciences (Scavone C, et al., 2020). A combination therapy using a drug cocktail of Remdesivir and Bepridil with a fixed concentration (7 μM) of BPD combined with various concentrations (0, 0.16, 0.31, and 0.62 μM) of RDV were used in a plaque reduction assays. The cocktails with the higher dose of RDV (0.62 μM) significantly increased inhibitory activity against SARS-CoV-2 infections (FIG. 4).

Administration of BPD (10 mg/kg) or TFP (10 mg/kg) in an animal model for SARS-CoV-2 infection should show inhibition of SARS-CoV-2 infection. In addition, oral formulations and other formulations of BPD (20 mg/kg) and/or TFP (20 mg/kg) alone or in combination with other antiviral drugs including Remdisivir, should inhibit SARS-CoV-2 infection.

### Compositions for Treating SARS-COV-2

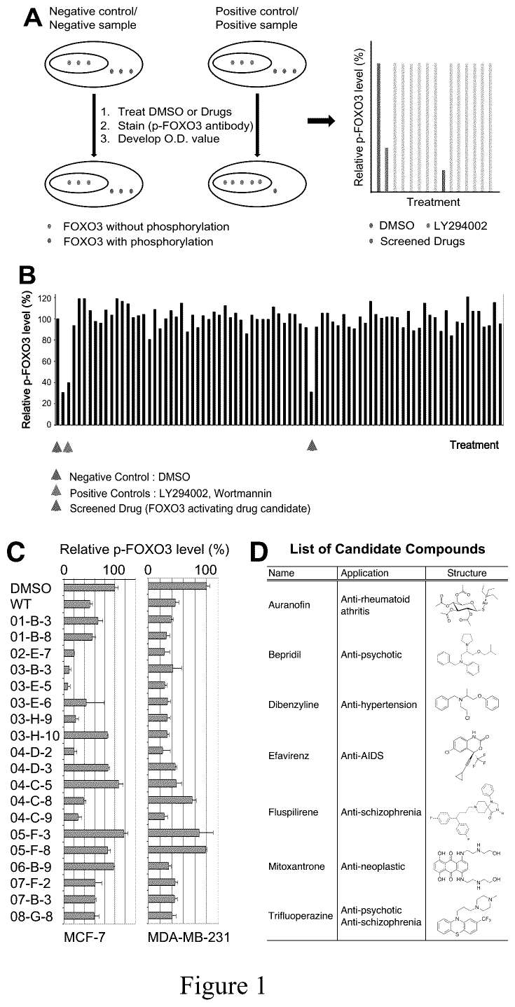

The compositions for treating SARS-COV-2 can include Bepridil and Trifluoperazine, whose compositions and structures are displayed in FIG. 1. Bepridil has anti-anginal and anti-arrhythmic actions by blocking calcium channels in heart and smooth muscle [Singh BN 1986; Perez-Reyes E, et al., 2009]. Trifluoperazine has anti-adrenergic and anti-dopaminergic actions typical of anti-psychotic drugs. Both Bepridil and Trifluoperazine) had been shown to antagonize the same protein, calmodulin, which acts as part of a calcium signal transduction pathway by modifying its interactions with various target proteins such as kinases or phosphatases [Stevens F C, 1983; Chin and Means, 2000].

Bepridil and Trifluoperazine can be combined in a composition with each other and/or with other antiviral compounds to make a cocktail or combination composition for treating SARS-CoV-2. The other antiviral drugs that can be combined with Bepridil and/or Trifluoperazine include, for example, Remdisivir, an anti-SARS-CoV-2 antibody that binds to the Spike 1 and/or 2 proteins, an antibody that neutralizes SARS-CoV-2, interferon-α, interferon-β, pegylated interferon-α, ribavirin, amantadine, rimantadine, zanamivir, oseltamivir, levovirin, viramidine, a nucleoside inhibitor of a retroviral polymerase inhibitor, an retroviral non-nucleoside polymerase inhibitor, an retroviral protease inhibitor, an retroviral helicase inhibitor or an retroviral fusion inhibitor. The antiviral can also include, for example, a drug that effects the biology of a virus and attenuates or inhibits attachment, entry, replication, shedding, latency or a combination thereof. The antiviral drug can also be a viral mimetic, a nucleotide analog, a sialidase inhibitor, or a protease inhibitor.

### Formulations and Methods of Administration

Therapeutic formulations of Bepridil and Trifluoperazine used in accordance with the present invention are prepared for storage by mixing Bepridil or Trifluoperazine having the desired degree of purity with optional pharmaceutically acceptable carriers, excipients or stabilizers (Remington's pharmaceutical sciences 18^{th} edition, Osol, A. Ed. (1990)), generally in the form of powdered formulations (as oral drugs) or organic (dimethyl sulfoxide) solutions (as intravenous drugs). Acceptable carriers, excipients or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride; phenol, butyl or benzyl alcohol; alkyl parabens such as methyl or propyl paraben; catechol; resorcinol; cyclohexanol; 3-pentanol; and m-cresol). Other inactive ingredients may include hydroxypropyl methyl-cellulose, lactose, magnesium stearate, microcrystalline cellulose, polyethylene glycol, silicon dioxide, pre-gelatinized corn starch, corn starch, titanium dioxide, FD&C Blue #1.

As oral drugs, the formulations for Bepridil drug can be as film-coated tablets comprising 200 or 300 or 400 mg of Bepridil hydrochloride with optional pharmaceutically acceptable carriers, excipients or stabilizers as described above in [0032]. The formulations for Trifluoperazine drug can be as film-coated tablets comprising 5 or 10 or 20 or 40 mg of Trifluoperazine hydrochloride with optional pharmaceutically acceptable carriers, excipients or stabilizers as described above in [0032].

The formulations can also include, for example, one or more suitable additive and/or auxiliary substance such as for example carrier materials, fillers, solvents, diluents, coloring agents and/or binders, surfactants, and may be administered as liquid medicament preparations in the form of injectable solutions, drops or juices, as semi-solid medicament preparations in the form of granules, tablets, pellets, patches, capsules, plasters or aerosols. The formulation can also include, for example, surfactants, fillers, such as pharmaceutical grade polyols or sugars (e.g., sucrose, sorbitol, mannitol, erythritol), binders (PVP, cellulose esters, polyethylene glycols), disintegrants (cross-carmellose, cross-povidone), preservatives (e.g., parabens, sorbic acid, benzoic acid and pharmaceutically acceptable salts thereof), lubricants, glidants, flavors, antioxidants, etc. The choice of the auxiliary substances, etc., as well as the amounts thereof to be used depend on whether the composition is to be administered orally, per orally, parenterally, intravenously, intraperitoneally, intradermally, intramuscularly, intranasally, buccally, rectally or topically, for example to the skin, the mucous membranes or the eyes. For oral application suitable preparations are in the form of tablets, sugar-coated pills, capsules, granules, droplets, juices and syrups, while for parenteral, topical and inhalative application suitable forms are solutions, suspensions, readily reconstitutable dry preparations, as well as sprays. The components can be incorporated into a tablet matrix, prepared by granulation, blending and compression. Compositions in a depot form, in dissolved form or in a plaster, optionally with the addition of agents promoting skin penetration, are suitable percutaneous application preparations. Preparation forms that can be administered orally or percutaneously can provide for the delayed release of the compositions described herein. In principle further active constituents known to the person skilled in the art may be added to the compositions described herein.

For treating animals in an animal model, the method of drug administration is either by feeding animals with Bepridil and Trifluoperazine by using oral gavage or an intravenous injection of Bepridil and Trifluoperazine.

For treating human COVID-19 diseases, the method of Bepridil and Trifluoperazine administration is taken by mouth (orally) as described above in [0033]. In general, food does not interfere with the absorption of Bepridil and Trifluoperazine.

Various features and embodiments of the disclosure are illustrated in the following representative examples, which are intended to be illustrative, and not limiting. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1 Inhibition of SARS-COV-2

The inhibitory effect of Bepridil (BPD) on SARS-CoV-2 infection of VeroE6 cells was measured as described in [0102]. Using a VeroE6 cell-based plaque reduction assay, BPD showed strong inhibition of SARS-CoV-2 infection in a dose-dependent manner (FIG. 2). The EC₅₀ value for BPD was 7.13 μM.

In the same manner, the inhibitory effect of Trifluoperazine (TFP) on SARS-CoV-2 infection of VeroE6 was measured in a plaque reduction assay. TFP potently inhibited SARS-CoV-2 infection in a dose-dependent fashion (FIG. 3). The respective EC₅₀ value for TFP was 7.7 μM.

Similarly, the effect of a combination therapy of Remdesivir (RDV) and BPD on SARS-CoV-2 infections was measured. Drug combinations were made using a fixed dose (7 μM) of BPD combined with various doses (0, 0.16, 0.31, and 0.62 μM) of RDV in a VeroE6 plaque reduction assay. The drug cocktails with higher doses of RDV (0.62 μM) and 7 μM BPD inhibited SARS-CoV-2 infection to a greater extent than BPD alone. The lower doses of RDV (0.16 and 0.31 μM) combined with BPD had no significant effect on inhibition activity beyond that obtained with BPD alone (FIG. 4).

### Example 2 Formulations of Bepridil and Trifluoperazine

As an oral drug, the formulations for Bepridil drug can be processed into film-coated tablets comprising 200 or 300 or 400 mg of Bepridil hydrochloride with optional pharmaceutically acceptable carriers, excipients or stabilizers as described above in [0032].

As an oral drug, the formulations for Trifluoperazine drug can be processed into film-coated tablets comprising 5 or 10 or 20 or 40 mg of Trifluoperazine hydrochloride with optional pharmaceutically acceptable carriers, excipients or stabilizers as described above in [0032].

### Example 3 Treatments with Bepridil and Trifluoperazine In Vivo

Using a mouse model system for SARS-CoV-2 infection, an effective dose of Bepridil and/or Trifluoperazine is administered to the mouse by an intravenous injection of Bepridil and/or Trifluoperazine. An ACE2 humanized C57BL/6 mice (human ACE2 CDS knocking into mouse Rosa 26 locus or human ACE2 replacing mouse ACE2) from Cyagen US Inc. is used foe this study. Equal numbers of female and male ACE2 humanized mice are used in these animal studies. Forty mice are randomly assigned to four different treatment groups, including control (DMSO), Bepridil, Trifluoperazine, and a combination of Bepridil and Trifluoperazine (5 female or male mice in each cage separately, total 10 mice per experimental group). The mice are infected with SARS-CoV-2 in saline solution with 10⁴ PFU intra-nasally using a nasal spray under ketamine/xylazine anesthesia. Twenty-four hours post-infection, the mice are treated with an intravenous injection (0.1 ml volume) of BPD (10 mg/kg) or TFP (10 mg/kg) or a combination of Bepridil and Trifluoperazine (10 mg/kg each) or DMSO (equal volume) once per week for 4 weeks. Body weight and pulmonary function of mice are monitored daily as a constant process once the viral infection is induced. After 5 weeks, mice are euthanized using CO2 or isoflurane overdose and tissue samples are harvested for titer and histopathology analysis. Mice used for nasal turbinate histopathology are perfused with 10% phosphate buffered formalin prior to tissue collection. Titer samples are stored at −80° C. until homogenization and titered by plaque assay as in [0013]. Histopathology samples are fixed in 10% phosphate buffered formalin for 7 days before paraffin embedding and sectioning. Slide sections are stained with hematoxylin and eosin (H&E) or used for immunohistochemistry for SARS-CoV-2 nucleocapsid staining with specific antibodies.

For oral administering to a subject infected with SARS-CoV-2 an effective dose of Bepridil and/or Trifluoperazine, liquid drugs are administered directly into the stomach of mice or rats via a technique called oral lavage. Bepridil or Trifluoperazine or a combination of both are dissolved in vehicle (10% DMSO in phosphate-buffered saline) at 0.25 or 0.5 mg of compound in 0.25 ml of vehicle (equivalent to 10 mg/kg or 20 mg/kg) in a 25-gram animal.

Using a primate model for SARS-CoV-2 infection, an effective dose of Bepridil and/or Trifluoperazine is administered to primates by an intravenous injection of Bepridil and/or Trifluoperazine as described above in [0043]. In general, human SARS-CoV-2 can infect experimental primates successfully without using the humanized animals expressing human ACE2.

For oral administering to a patient infected with SARS-CoV-2 an effective dose of Bepridil and/or Trifluoperazine in a human therapy, these drugs can be used as tablets as described above in [0032 and 0033].

### Example 4 Other Biological Activities of Bepridil and Trifluoperazine

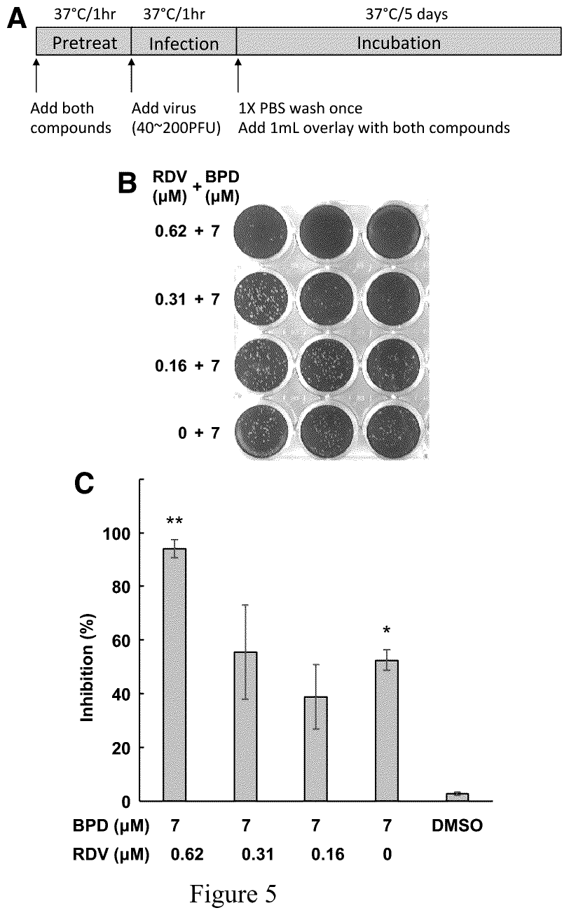

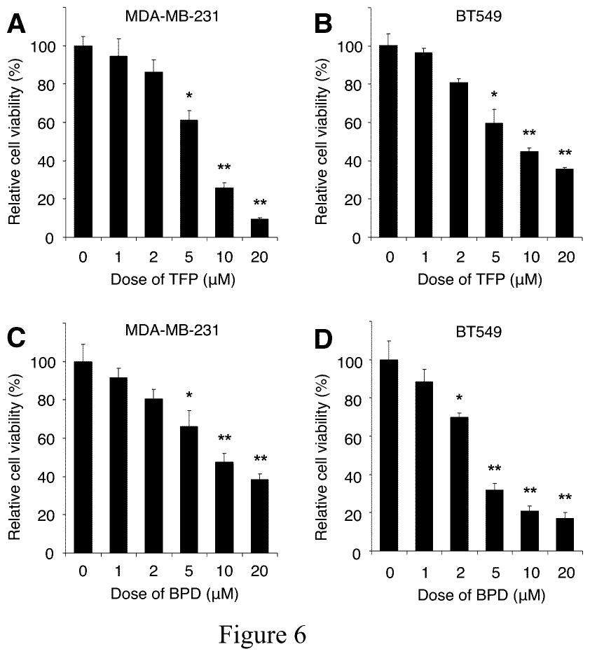

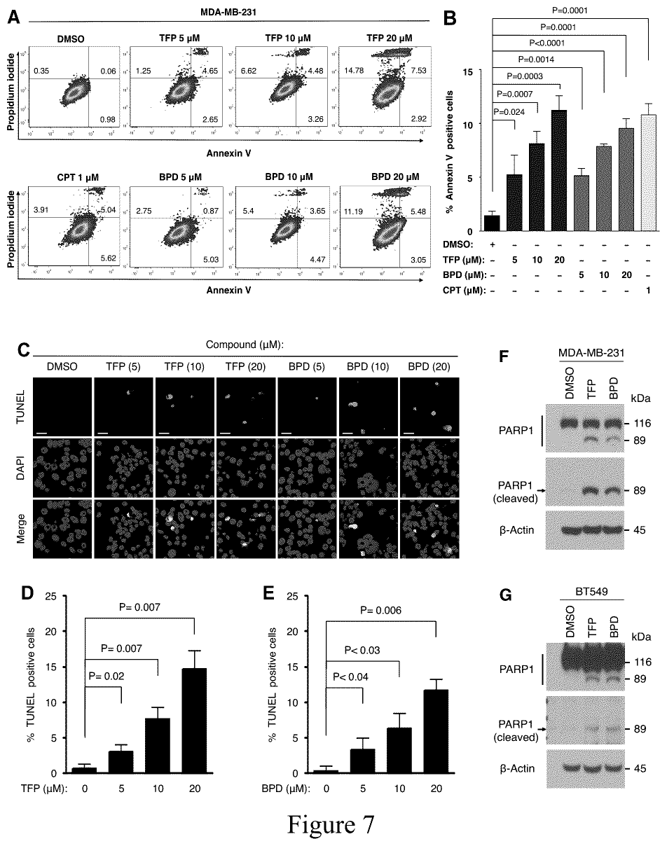

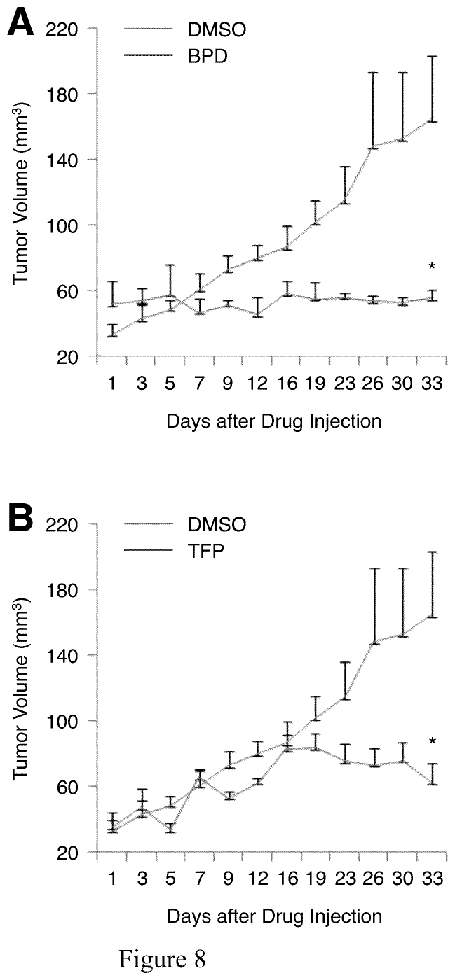

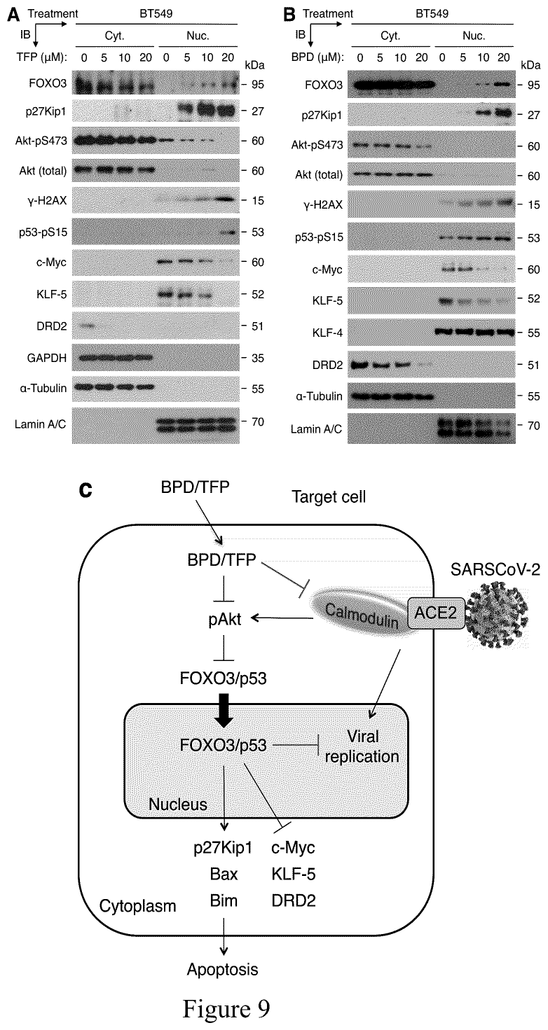

We have proposed a schematic representation to display that BPD and TFP inhibit calmodulin and lead to suppression of SARS-CoV-2 binding to ACE2 on the surface of target cells. In addition, BPD and TFP suppress phosphor-Akt (pAkt) and induce FOXO3 and p53 (FOXO3/p53) translocation from the cytoplasm into the nucleus, where activated p53-pS15 inhibits SARS-CoV-2 viral replication and results in inhibition of SARS-CoV-2 infection (Figure. 5).

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the scope of the invention(s) of the disclosure.

### REFERENCES

Chattopadhyay S, Santhamma K R, Sengupta S, et al. Calmodulin binds to the cytoplasmic domain of angiotensin-converting enzyme and regulates its phosphorylation and cleavage secretion. J Biol Chem. 2005; 280(40):33847-33855.
Chen L W, Jan C R. Effect of the antianginal drug bepridil on intracellular Ca2+ release and extracellular Ca2+ influx in human neutrophils. Int Immunopharmacol. 2001; 1(5):945-953.
Chong C R, Sullivan D J Jr. New uses for old drugs. Nature. 2007; 448(7154):645-646.
Chung Y M, Park S H, Tsai W B, Wang S-Y, Ikeda M-A, Berek J S, Chen D J, Hu M C. and FOXO3 signalling links ATM to the p53 apoptotic pathway following DNA damage [published correction appears in Nat Commun. 2013; 4:1520]. Nat Commun. 2012; 3:1000.
Colbourn T. COVID-19: extending or relaxing distancing control measures. Lancet Public Health. 2020; 5(5):e236-e237.
Cowling B J, Ali S T, Ng T W Y, et al. Impact assessment of non-pharmaceutical interventions against coronavirus disease 2019 and influenza in Hong Kong: an observational study. Lancet Public Health. 2020; 5(5):e279-e288.
de Wit E, van Doremalen N, Falzarano D, Munster V J. SARS and MERS: recent insights into emerging coronaviruses. Nat Rev Microbiol. 2016; 14(8):523-534.
Donoghue M, Hsieh F, Baronas E, et al. A novel angiotensin-converting enzyme-related carboxypeptidase (ACE2) converts angiotensin I to angiotensin 1-9. Circ Res. 2000; 87(5):E1-E9.
Fu Z, Tindall D J. FOXOs, cancer and regulation of apoptosis. Oncogene. 2008; 27(16):2312-2319.
Gu Y, Zhang J, Ma X, et al. Stabilization of the c-Myc Protein by CAMKIIγ Promotes T Cell Lymphoma. Cancer Cell. 2017; 32(1):115-128.e7.
Huang C, Wang Y, Li X, et al. Clinical features of patients infected with 2019 novel coronavirus in Wuhan, China [published correction appears in Lancet. 2020 Jan. 30;:]. Lancet. 2020; 395(10223): 497-506.
Kindrachuk J, Ork B, Hart B J, et al. Antiviral potential of ERK/MAPK and PI3K/AKT/mTOR signaling modulation for Middle East respiratory syndrome coronavirus infection as identified by temporal kinome analysis. Antimicrob Agents Chemother. 2015; 59(2):1088-1099.
Lambert D W, Clarke N E, Hooper N M, Turner A J. Calmodulin interacts with angiotensin-converting enzyme-2 (ACE2) and inhibits shedding of its ectodomain. FEBS Lett. 2008; 582(2):385-390.
Li W, Moore M J, Vasilieva N, et al. Angiotensin-converting enzyme 2 is a functional receptor for the SARS coronavirus. Nature. 2003; 426(6965):450-454.
Ma-Lauer Y, Carbajo-Lozoya J, Hein M Y, et al. p53 down-regulates SARS coronavirus replication and is targeted by the SARS-unique domain and PLpro via E3 ubiquitin ligase RCHY1. Proc Natl Acad Sci USA. 2016; 113(35):E5192-E5201.
Mizutani T, Fukushi S, Saijo M, Kurane I, Morikawa S. Importance of Akt signaling pathway for apoptosis in SARS-CoV-infected Vero E6 cells. Virology. 2004; 327(2):169-174.
Park S H, Chung Y M, Ma J, Yang Q, Berek J S, Hu M C. Pharmacological activation of FOXO3 suppresses triple-negative breast cancer in vitro and in vivo. Oncotarget. 2016; 7(27):42110-42125.
Park S H, Lee J H, Berek J S, Hu M C. Auranofin displays anticancer activity against ovarian cancer cells through FOXO3 activation independent of p53. Int J Oncol. 2014; 45(4):1691-1698.
Prem K, Liu Y, Russell T W, et al. The effect of control strategies to reduce social mixing on outcomes of the COVID-19 epidemic in Wuhan, China: a modelling study [published correction appears in Lancet Public Health. 2020 May; 5(5):e260]. Lancet Public Health. 2020; 5(5):e261-e270.
Prieto A, Diaz D, Barcenilla H, et al. Apoptotic rate: a new indicator for the quantification of the incidence of apoptosis in cell cultures. Cytometry. 2002; 48(4):185-193.
Reiermann H J, Rüegg JC. Investigations on calmodulin antagonistic effects of bepridil in intact and skinned fibres of smooth muscle. Arzneimittelforschung. 1986; 36(4):668-670.
Scavone C, Brusco S, Bertini M, et al. Current pharmacological treatments for COVID-19: What's next? [published online ahead of print, 2020 Apr. 24]. Br J Pharmacol. 2020; 10.1111/bph.15072.
Soldani C, Lazzè MC, Bottone M G, et al. Poly(ADP-ribose) polymerase cleavage during apoptosis: when and where?. Exp Cell Res. 2001; 269(2):193-201.
Tardy M, Dold M, Engel R R, Leucht S. Trifluoperazine versus low-potency first-generation antipsychotic drugs for schizophrenia. Cochrane Database Syst Rev. 2014; (7):CD009396.
Tipnis S R, Hooper N M, Hyde R, Karran E, Christie G, Turner A J. A human homolog of angiotensin-converting enzyme. Cloning and functional expression as a captopril-insensitive carboxypeptidase. J Biol Chem. 2000; 275(43):33238-33243.
Winkler M A, DeWitt L M, Cheung W Y. Calmodulin and calcium channel blockers. Hypertension. 1987; 9(3):217-223.
Zhang M, Jang H, Gaponenko V, Nussinov R. Phosphorylated Calmodulin Promotes PI3K Activation by Binding to the SH₂ Domains. Biophys J. 2017; 113(9):1956-1967.
Zimmer M, Hofmann F. Calmodulin antagonists inhibit activity of myosin light-chain kinase independent of calmodulin. Eur J Biochem. 1984; 142(2):393-397.

Each of the foregoing references is incorporated by reference in its entirety for all purposes.

1. A method comprising the steps of: administering to a subject infected with SARS-CoV-2 a therapeutically effective amount of a pharmaceutical composition comprising a therapeutically effective amount of an antiviral agent, wherein the antiviral agent is a Bepridil, a Trifluoperazine or a pharmaceutically acceptable salt thereof, and a pharmaceutically acceptable carrier.

2. The method of claim 1, wherein the antiviral agent is a Bepridil.

3. The method of claim 1, wherein the antiviral agent is a Trifluoperazine.

4. The method of claim 1, wherein the antiviral agent further comprises another therapeutic agent.

5. The method of claim 4, wherein the antiviral agent is a mixture of a Bepridil and a Remdesivir.

6. The method of claim 4, wherein the antiviral agent is a mixture of a Trifluoperazine and a Remdesivir.

7. The method of claim 4, wherein the antiviral agent is a mixture of a Bepridil and a Trifluoperazine,

8. The method of claim 1, wherein the pharmaceutical composition further comprises an analgesic, a decongestant, an anti-inflammatory, or an immunosuppressive agent.

9. The method of claim 1, wherein the pharmaceutical composition is an oral dosage form.

10. The method of claim 1, wherein the pharmaceutical composition is formulated as an injection dosage form.

11. A method for prophylactically treating a subject, comprising the steps of: administering to the subject a therapeutically effective amount of a pharmaceutical composition comprising a therapeutically effective amount of an antiviral agent, wherein the antiviral agent is a Bepridil, a Trifluoperazine or a pharmaceutically acceptable salt thereof, and a pharmaceutically acceptable carrier.

12. The method of claim 11, wherein the antiviral agent is a Bepridil.

13. The method of claim 11, wherein the antiviral agent is a Trifluoperazine.

14. The method of claim 11, wherein the antiviral agent is a mixture of an antiviral drug and another therapeutic agent.

15. The method of claim 11, wherein the antiviral agent is a mixture of an antiviral drug and a second antiviral drug.

16. The method of claim 11, wherein the antiviral agent is a mixture of a Bepridil and a Remdesivir.

17. The method of claim 11, wherein the antiviral agent is a mixture of a Trifluoperazine and a Remdesivir.

18. The method of claim 11, wherein the antiviral agent is a mixture of a Bepridil and a Trifluoperazine,

19. The method of claim 11, wherein the pharmaceutical composition further comprises an analgesic, a decongestant, an anti-inflammatory, or an immunosuppressive agent.

20. The method of claim 11, wherein the pharmaceutical composition is an oral dosage form.

21. (canceled)

22. (canceled)

23. (canceled)

24. (canceled)
